(19) United States
(12) Patent Application Publication
(10) Pub. No.: US 2019/0083395 A1
(43) Pub. Date: Mar. 21, 2019

(54) BREATHING ENHANCEMENT DEVICE

(71) Applicant: Navin Doshi, Los Angeles, CA (US)

(72) Inventor: Navin Doshi, Los Angeles, CA (US)

(21) Appl. No.: 16/019,425

(22) Filed: Jun. 26, 2018

Related U.S. Application Data
(60) Provisional application No. 62/561,025, filed on Sep. 20, 2017.

(51) Int. Cl. A61K 9/00; A61M 11/04; A61M 15/00

(52) U.S. Cl. CPC A61K 9/0078; A61K 33/14; A61M 15/009; A61M 11/04

(57) ABSTRACT

A breathing device is formed of a container having a spherically-shaped cavity in which a medicinal substance is located. The cavity has input and output ports. Pressurized and temperature-controlled breathable air is provided at the input to the cavity with enough pressure so that the air circulates through the medicinal substance to infuse the medicinal substance into the air. A fluid permeable bag is used to hold salt crystals, such as Himalayan pink salt, in the cavity. A medicinal oil is added to the salt crystals. An outlet port is formed in the container and is connected with a vented breathing mask for a user to inhale the salt- and oil-infused air. The pressure of the breathable air at the input is strong enough to circulate the air through the salt crystals and oil and expel the air from the cavity through the output port to provide positive pressure at a vented breathing mask.

### CROSS-REFERENCE TO RELATED APPLICATIONS

This application claims the benefit of U.S. provisional application no. 62/561,025 filed Sep. 20, 2017, the disclosure of which is hereby incorporated by reference.

### TECHNICAL FIELD

The present invention relates to devices for administering medicinally infused air to a user of the device. More particularly, the present invention relates to admixing an air stream with salt particles and therapeutic oil to form an aerosol and delivering that aerosol to be breathed by a user to relieve respiratory and other disorders.

### BACKGROUND

Respiratory diseases are a global problem. Many people worldwide are afflicted with these medical conditions. They affect both adults and children. These respiratory diseases, which include asthma, chronic obstructive pulmonary disease (“COPD”), chronic sinusitis, and cystic fibrosis, reduce the quality of life and impair the ability of sufferers to perform everyday tasks. Some people are so strongly affected that they cannot contribute to the work force.

Asthma is a disease in which the bronchi are inflamed, narrowed, and obstructed. This narrowing of bronchi results from a combination of bronchial muscle contraction, mucosal edema, inflammatory cell infiltrate, and partial or total occlusion of the lumina with mucus, cells, and cell debris. Bronchial obstruction is either partially or totally reversible. Asthma has become more common worldwide. In the developed world it is one of the most common chronic illnesses. In susceptible individuals this inflammation causes symptoms that are usually associated with widespread, but variable, airflow obstruction. This is often reversible, either spontaneously or with treatment, and causes an associated increase in airway responsiveness to a variety of stimuli.

“COPD” is a term used for chronic airway obstruction. As written above, it stands for “chronic obstructive pulmonary disease.” COPD is considered a non-specific term because it covers two subsets: chronic bronchitis and emphysema. It also refers to chronic obstructive airways disease, chronic obstructive lung disease, and “smoker's chest.” COPD is characterized by progressive and irreversible airway obstruction. It can lead to death from respiratory or cardio-respiratory failure. The present treatment of COPD consists of bronchodilators, intermittent courses of antibiotics and, in some patients, inhaled and/or oral corticosteroids. The latter are claimed to reduce the decline in lung function in COPD sufferers.

Corticosteroids have been the mainstay of COPD and asthma treatment for many years. There are two ways of administering corticosteroids, inhaled and oral, both of which require a doctor's prescription. Inhaled corticosteroids are often used for treatment although severe asthmatics still require medication by mouth (oral). Inhaled corticosteroids are relatively safe and extremely effective in most patients, and have improved the quality of life for millions of asthma sufferers. For those with severe asthma, however, oral therapy with corticosteroids is required. However, when taken for more than a few days, oral corticosteroids have a number of serious side effects. Although corticosteroids are often effective, they are not ideal drugs. Over the years doctors have occasionally used immunosuppressive agents as adjuncts to corticosteroids in patients with extremely severe disease.

Halotherapy is an alternative treatment that involves breathing salty air. It has been found to relieve respiratory conditions, such as asthma, chronic bronchitis, and allergies. Halotherapy is usually broken down into dry and wet methods, depending on how the salt is administered.

The commonly-known “dry” method of halotherapy is usually done in a man-made “salt cave” that is free of humidity. The temperature is kept cool at approximately 20° C. (68° F.). Salt is ground into microscopic particles and is released into the air of the room. Once inhaled, it is believed that these microscopic salt particles absorb irritants, including allergens and toxins, from the respiratory system of the patient. The microscopic salt particles break up mucus and reduce inflammation, resulting in clearer airways.

Wet halotherapy is usually done by mixing salt and water and includes using salt for nasal irrigation and gargling with salt water as two examples. The growth of fungus and other microorganisms is a concern with wet halotherapy. Cleaning of equipment and all surfaces involved must be performed. These needs for cleaning, the inconvenience involved, and the chance for ancillary infections due to fungus, mold, or other microbes make wet halotherapy less desirable and dry halotherapy preferred.

Dry halotherapy has been practiced for centuries. A well-known natural dry halotherapy treatment is to spend time in a salt mine. The Wieliczka Salt Mine in southern Poland is one of the oldest operating salt mines in Europe. The mine has been in operation for over eight-hundred years and has more than one-hundred and twenty miles of passageways and chambers on nine levels to a depth of more than one-thousand feet. It is used as a sanatorium for people who have bronchial and allergic asthma. Patients live on the surface and are lowered into the mine each day for six hours where they breathe soothing salt air. This treatment has had positive effects on many patients over many years. (See https://health-resort.wieliczka-saltmine.com/underground-treatment/paid-stays/health-day)

In recent times, dry halotherapy has been noted as a powerful drug-free treatment for patients with chronic respiratory ailments. When a natural salt mine or cave is not available, special rooms are created to simulate the atmosphere of the interior of a salt mine. Such rooms are often very effective in treating patients for various respiratory ailments. Nevertheless, monetary restraints and lack of access to such caves and rooms for other reasons limit most patients from availing themselves of the healing salt air of natural salt mines or man-made salt rooms. A need for a lower-cost and more accessible portable dry-halotherapy device exists to treat respiratory patients wherever they may be.

In practicing halotherapy, a certain concentration of salt in the breathing air is necessary. Too little salt will not have the desired therapeutic effects. Too much salt tends to irritate the mucus membranes of the respiratory passages and thus negate the desired relief and beneficial effect and, in fact, may aggravate the condition. A correct amount of salt is important to obtain beneficial effects. Any portable device for administering halotherapy to a sufferer must be carefully designed to control the amount of salt in the breathed air so that subjecting the patient to too much or too little salt can be avoided. Consistency in the salt concentration from one treatment to the next by the portable device is also desired.

Providing halotherapy to the lower respiratory tract of a patient has included the use of operating a plastic device and inhaling air through that plastic device. A part of the plastic device contains the breathable salt. The patient operates the plastic device to release a dose of salt-infused air for inhalation when the apparatus is aimed into the user's mouth. The user then deeply inhales the dose of salt-infused air to target his or her lower respiratory tract (lungs). This inhaled dose of salt-infused air is meant to supply a salt concentration to produce a halotherapy effect in the lungs to avoid the negative effects of continual use of corticosteroids.

While this dry halotherapy technique can be very helpful to many who have respiratory afflictions, charging the inhalation device with the necessary salt and obtaining a constant concentration of salt in the inhaled air from treatment to treatment can be difficult. Some portable devices do not have a consistent and uniform air flow through the device and across the salt which results in the possibility of the concentration of salt in the breathable air varying from treatment to treatment. Discarding old, no longer effective salt and replacing it with new salt also can be difficult. In some present inhalation devices, a disposable salt device cartridge is used. This disposable cartridge may take the form of specially-shaped disks of salt, or predesigned disposable containers that fit in specially-shaped receptacles in the inhalation device. If the user of the inhalation device were to use his or her entire supply of these specially designed cartridges of salt devices, the inhalation device would not be functional until new cartridges can be obtained. There is therefore a need for a treatment device that can accept quantities of salt in its internal container that do not need to be placed in any particularly-designed cartridge.

It would be desirable to have an inhalation device that did not depend on specially-designed container shapes as replacement salt cartridges. It would be desirable if an inhalation device were designed to use a wide variety of salts without the need for any particular shape of container for the salt that goes into the inhalation device. In doing so, the salt container should be shaped for easy control over the concentration of salt in the breathed air. In particular, the inhalation device should have an air flow design through them that causes the efficient circulation of breathing air through the entire volume in which salt is stored for admixing efficiently the salt with the stream of breathing air. The internal “flow path” of the breathing air through the volume in which the salt is stored should be conducive to moving the air through the salt to obtain an aerosol having the desired concentration of salt and not an excessive amount, which can irritate the patient. Providing a well-designed salt container that causes air flow patterns through the salt container to be consistent, uniform, and covering the entire storage area is desired.

Cold viruses find the temperature within the nose, which is about 33° C. (91.4° F.), more agreeable than the warmer climate of the blood and internal organs, which are at 37° C. (98.6° F.). Cold viruses attack the cells of the mucous membrane, producing congestion, sneezing and nasal drip. Some viruses have other effects, including aches, fever, coughing, and chills. The microbes that cause the common colds take two to three days to incubate and can take one to two weeks to run their course, declining slowly from an early peak. Sufferers are most infectious at the beginning, when sneezing and dripping are at their height. The virus kills the nasal cells it infects, and it takes time to regenerate them. This is one explanation of why it may take a while to recover from a cold.

It has been found that artificially increasing the temperature of the nasal passages in which cold viruses are resident to a temperature above 37° C. (98.6° F.) can kill or seriously weaken the cold virus. Temperatures above 41° C. (106° F.) are even more likely to kill or significantly harm cold-causing microbes. Many medications have been found to be ineffective against the cold virus but it has been found that a stream of warmed, salt-infused air can decrease the effects of the common cold, COPD, and asthma. It would be desirable to provide a source for a stream of warm and salt-infused air to treat both COPD and any nasal infections that exist. As with the discussion above of halotherapy in which the ability to control the amount of salt in the air is important, it is also very desirable to control the amount of heat in the breathing air so that a hyperthermia level is obtained in the user.

Combining a stream of warmed air with a concentration of salt infusion would be desirable for treating a patient who has both a respiratory affliction and the possibility of contracting another respiratory infection, such as a cold. Various prior art devices have been proposed for the treatment of respiratory or infectious problems with warm air but typically involve problems with controlling the temperature of the air and controlling the concentration of salt in the air. Also causing difficulty is the prior design for replacing salt in the container that is used for creating the breathing aerosol.

Those involved in the arts of halotherapy have recognized a need for a portable, more accessible inhalation device that can not only provide a salt-infused aerosol having a consistent salt amount to a user for breathing into his or her lungs, but also a device where the temperature of that salt-infused aerosol may be set at a hyperthermia level to attempt to neutralize microbes that cause colds. Hyperthermia, as it is used herein, has come to mean temperatures in humans above 41° C. (106° F.).

Essential oils have been used therapeutically for centuries. Their use is often referred to as “aromatherapy.” Therapeutic benefits attributed to essential oils range from mood elevation and stress relief to remedies for chronic pain, insomnia, migraine, asthma, COPD, arthritis, and others.

Essential oils are the distilled essence of various substances and are often made from herbs of some kind, but are also made from other plants. An essential oil is made by condensing the potent effects of the plant into a single liquid form. Essential oils are very potent, so much so that they can be difficult to use. Examples of essential oils are lavender, peppermint, sage, dill seed, eucalyptus, lemon, rosemary, spearmint, and frankincense. There are many others.

Not all essential oils can be inhaled safely, but many which can be inhaled have been found to produce beneficial effects in the user. Essential oils inhaled through the nose first pass through the olfactory system, which includes physical organs or cells contributing to the sense of smell. When essential oils are inhaled through the nose, airborne molecules interact with the olfactory organs and, almost immediately, the brain. From there, the inhaled essential oils pass down the trachea into the bronchi and from there into finer and finer bronchioles, ending at the microscopic, sac-like alveoli of the lungs, where gaseous exchange with the blood takes place. The alveoli are efficient at transporting small molecules, such as essential oil constituents, into the blood. This efficiency increases with the rate of blood flow through the lungs, the rate and depth of breathing, and with the fat-solubility of the molecules. Essential oil constituents absorbed via inhalation enter the bloodstream and then reach various parts of the body. Molecules inhaled through the nose are carried to the lungs, interact with the respiratory system, and then enter the circulatory system.

Essential oils are very volatile. They react with oxygen in the air and evaporate quickly. This provides a benefit in that it is the reason that why the oil gets into the air for breathing; however, this volatility makes them very strong and very short-acting. In order to make them safe for topical use and to extend their viability, they work better with a different form of administration. It is common to use a “carrier oil” to make the essential oil less volatile. Examples of carrier oils are coconut oil, olive oil, and grapeseed oil. There are others. The carrier oils are non-reactive with essential oils and mix well with them. They allow the essential oil to evaporate much more slowly, and dilute the potency of the oil so that it is less damaging in concentration. However, using carrier oils involves additional expense and the effort of blending the carrier and essential oils together can be messy and inaccurate.

A common administration of essential oils to a user is simply to open the top of the container of the essential oil, bring it close to the nose, and breathe in. The user will experience some of the scent but this is not a controlled administration. The user does not experience a full impact of undiluted essential oil. This administration requires the user to breathe in to draw the scent into his or her lungs. Depending on the user's lung power, the scent may or may not reach deeply into the lungs. Additionally, the user must avoid snorting the oil, which can be harmful. A common warning with essential oils is to avoid applying these oils directly to the nostrils or to the eyes, or to any other mucous membrane because harm can result.

Another form of administration of essential oils is diffusion of the oil into the breathable air. A common approach to diffusion is applying essential oil to cotton balls in a bowl and inhaling the aroma from just above the bowl of oil. Unfortunately, the effectivity of the oil dissipates rapidly due to the volatile nature of the oils. Also, this is not an accurate way to administer the oils. This administration requires the user to breathe in to draw the scent into his or her lungs. Depending on the user's lung power, the scent may or may not reach deeply into the lungs.

Steam inhalation provides more absorption of essential oils than other methods. This involves heating water in a pan until it is steaming and adding 1-2 drops of essential oil to the water. The user often puts a towel over his or her head located above the steaming water, and inhales the essential oil that is transported with the steam rising from the pan of steaming water. This has been found to successfully open up the sinuses and help relieve respiratory congestion. It can also be used as a bronchitis and asthma remedy. However, the user is cautioned to keep his or her eyes closed, as some essential oils can cause a burning sensation to the eyes. This administration requires the user to breathe in to draw the scent into his or her lungs. Depending on the user's lung power, the scent may or may not reach deeply into the lungs. While some relief can be experienced with this time-honored approach to respiratory relief, steaming water, taking care in not getting burned, and cleaning up afterward are all inconvenient. Cleanliness is required because this is a “wet” approach and fungus and harmful bacteria can form in uncleaned apparatus. However, an advantage of this approach is that the essential oil or oils are simply dropped into the steaming water. No mixture with carrier oils or cotton balls is required.

Nebulizers are also used for inhalation of essential oils and can be effective. A nebulizer is a machine that converts a cold liquid into a vapor for breathing. It is a wet system and they are often used to administer medicines; however, in order to obtain vaporization of the essential oil, they spray it into the air in the environment. These devices also require cleaning and are an inefficient means to administer the essential oils. Due to the volatility of the oils, they are gone rapidly. These devices rely on the strength of the user's lungs to draw the scent into his or her lungs. Depending on the user's lung power, the scent may or may not reach deeply into the lungs.

Portable aromatherapy diffusers can be found in many stores and are often single use, disposable devices. Reusable ones are more complicated and still require cartridges or another form that is disposable.

While there is little science supporting the benefits of aromatherapy, there is common belief that it does have therapeutic benefits. It would be an advantage to provide a useful aromatherapy device that is easy to use, is simplistic in using the essential oils, and that can be inhaled directly by a user.

In the above-discussed approaches, the flow of medicated breathing air into a user's lungs is dependent on the lung strength of the user. This can be based on the user's lung muscle strength. If a user has low lung strength, the salt and oil-infused air may not go deeply into the lungs. Having a positive pressure breathing device in which the aerosol breathing stream of air is forced into the user's lungs is desirable. In such a system, the user merely performs an easy breathe-in maneuver and the positive pressure breathing device will force the aerosol airstream deeply into the user's lungs. When the user is ready to exhale, he or she may do so.

A deficiency in consistent and uniform air flow has been noted in portable halotherapy and aromatherapy devices. The flow distribution of the air through the containers of such devices in which the salt or oil is located is not uniform. This can result in inconsistent administrations of the admixed medicinal substance or substances located within the container. This is especially true for those devices where the air flow is solely caused by the user's inhalation lung power. For those users who do not have strong inhalation strength, the concentration of salt and oil in the air flow may have a lower concentration. However, even for those with strong lung inhalation power, the concentration of salt and oil in the aerosol may vary due to the design of the device where the air flow does not reach all the medicinal substances. Those of skill in the art have noted that the internal shape of a container in which salt and oil reside and through which the breathing air flows, and the placement of the air input port and air output port can affect the circulation of the air flow inside the device, thereby affecting the concentration of salt and oil in the aerosol. If the container is designed poorly, the air flow may not reach every salt crystal or every source of oil inside the container.

Hence, those of skill in the art have recognized a need for a means to provide more consistent administrations of breathable air admixed with medicinal substances. A need has also been recognized for a device that provides a means to control the temperature of breathable air that has been mixed with salt in the administration to a user. A further need has been recognized to provide an administration device configured to provide breathable air that comprises infusions of salt and oil. Yet another need has been identified for a device that provides both halotherapy and aromatherapy simultaneously, but which is easier to use, preserves the essential oils for controlled use, and is easy to reload with new salt and essential oil. Another need has been recognized for a positive pressure breathing device to administer more efficiently and deeply the medicated airstream into the user's lungs. The present invention fulfills these needs and others.

### SUMMARY OF THE INVENTION

Briefly and in general terms, the present invention is directed to a breathing enhancement device for administering medicinally-infused and temperature-controlled air into the lungs of a user under positive pressure. The use of positive pressure and a spherically-shaped cavity in which a medicinal substance or substances are place results in excellent circulation of breathable air through the medicinal substance for infusion of the medicinal substance into the breathable air. The use of positive pressure forces the infused air into the lungs of a user. A vented mask permits the user to exhale during the application of the medicinally-infused air under positive pressure.

In other aspects in accordance with the invention, there is provided a breathing device for administering medicinally-infused air into the lungs of a user, the breathing device comprising a container with an outside surface that defines an inner cavity, and an inner surface within the cavity, the inner cavity being generally spherically-shaped with a size selected for receiving a medicinal substance, the container further comprising separate input and output ports a vented inhalation mask having a hollow mask connection tube connected to the container output port to receive air flowing out of the container and conduct the air to the mask for inhalation by a user and an air pressurization device configured to receive breathable air and to pressurize the received breathable air, the air pressurization device having a pressurized air output port that is connected to the input port of the container through which the pressurized breathable air is introduced to the inner cavity of the container to circulate the pressurized air through the medicinal substance located within the cavity to infuse the medical substance into the pressurized air as it is circulated through the medicinal substance, and to expel the circulated, breathable, and medicinally-infused air out of the cavity through the output port of the container.

In other, more detailed aspects, the breathing device further comprises a hollow air circulation tube having a first end, a second end, and a length, the first end of the air circulation tube being connected within the cavity to the input port of the container to receive the pressurized air, the air circulation tube having a length selected to position the second end of the circulation tube farther into the cavity than the output port of the container, whereby pressurized air introduced to the cavity at the input port of the container is conducted by the air circulation tube deeper into the cavity than the location of the output port of the container so that the pressurized air is circulated through the medicinal substance located in the cavity before reaching the output port of the container and being expelled to the vented inhalation mask. The input port of the container has an opening that is larger than an opening of the output port of the container to cause resistance to flow of the pressurized breathable air through the cavity resulting in greater circulation of the pressurized input air through the medicinal substance in the cavity prior to the pressurized air being expelled through the output port of the container to the inhalation mask for inhalation by a user. In a different aspect, the locations of the input port of the container and the output port of the container are opposite each other in relation to the cavity.

In further aspects, the breathing device further comprises an air temperature control device configured to adjust the temperature of the breathable air that is provided to the input port of the container. In another detail, the air temperature control device comprises a heater positioned to heat the breathable air before the air enters the cavity through the container inlet port.

In other aspects, the breathing device further comprises a medicinal substance within the cavity that comprises salt crystals, and wherein the pressurized breathable air introduced through the input port of the container circulates among the salt crystals before being expelled from the cavity through the output port of the container to the inhalation mask for inhalation by the user. In another aspect, the medicinal substance within the cavity further comprises a medicinal oil, and wherein the pressurized breathable air introduced to the cavity through the input port of the container is caused to circulate among the salt crystals and the medicinal oil before being expelled through the output port of the container to the inhalation mask for inhalation by the user. In yet another aspect, the medicinal substance within the cavity comprises cannabinoids, and wherein the pressurized breathable air introduced to the cavity through the input port of the container is caused to circulate among the cannabinoids before being expelled through the output port of the container to the inhalation mask for inhalation by the user.

The breathing device comprises a first fluid-permeable bag containing salt crystals, the first bag of salt crystals being located within the cavity of the container wherein the pressurized breathable air introduced through the input port of the container is caused to circulate through the first fluid-permeable bag and among the salt crystals therein before being expelled through the output port of the container to the inhalation mask for inhalation by the user. In a different aspect, the breathing device further comprises a second fluid-permeable bag containing a medicinal substance, the second bag of the medicinal substance being located within the cavity of the container adjacent the first bag wherein the pressurized breathable air introduced through the input port of the container circulates through the first fluid-permeable bag comprising salt crystals and through the second fluid-permeable bag of medicinal substance before being expelled through the output port of the container to the mask for inhalation by the user.

Related to the above, the breathing device further comprises an opening formed through the outer surface of the container, the opening having a size large enough to place the fluid-permeable bag in the cavity and to remove the fluid-permeable bag from the cavity, and a cover removably positioned over the opening, the cover configured to resist passage of pressurized breathable air out of the cavity through the opening. In additional aspects, the input and output ports of the container are provided in the cover, and wherein the cover is configured to resist passage of pressurized breathable air out of the cavity through the opening. In a further aspect, the container is formed of a coconut shell. Coconut oil is coated onto the inner surface of the cavity of the coconut.

Further aspects include a breathing device comprising a container having a generally spherically-shaped internal cavity configured to receive an infusible medicinal substance, the container having an inlet in fluid communication with the spherically-shaped internal cavity and an outlet in fluid communication with the spherically-shaped internal cavity, a breathing mask externally secured to the container and in fluid communication with the outlet of the cavity, and an air pressurization device secured to the container and configured to produce pressurized breathable air and to force the pressurized breathable air through the inlet into the spherically-shaped internal cavity to flow across an infusible medicinal substance located in the cavity so that the medicinal substance is infused into the pressurized air flowing across it, and to expel the infused breathable air through the outlet and into the breathing mask.

Method aspects include a method of providing medicinally-infused breathable air, comprising pressurizing breathable air, applying the pressurized breathable air to an input port of a container that has a spherically-shaped interior cavity in which is located an infusible medicinal substance, flowing the pressurized breathable air through the input port and across the medicinal substance to infuse the flowing pressurized breathable air with the medicinal substance, expelling the infused pressurized breathable air from the cavity through an output port of the cavity, and directing the expelled infused pressurized breathable air to a vented face mask. Additional method aspects include heating the breathable air prior to applying the pressurized breathable air to the input port of the container. Flowing the pressurized breathable air across salt crystals located in the spherically-shaped interior cavity of the container. Disposing a medicinal substance within a fluid-permeable bag and then locating the fluid permeable bag within the spherically-shaped interior cavity.

The features and advantages of the invention will be more readily understood from the following detailed description that should be read in conjunction with the accompanying drawings.

### BRIEF DESCRIPTION OF THE DRAWINGS

FIG. 1 is a partially cutaway side view of a first embodiment of a breathing enhancement device for administering air into the lungs of a user that has been infused with a medicinal substance, showing a vented inhalation face mask at the left, an admix container in the middle in which medicinal substances are mixed, or infused, with an air flow to form an aerosol, and in block form an air flow control unit to the right side that provides pressurized and heated air to the container;

FIG. 2 is a perspective view looking downward at the top and side of a second embodiment of a breathing enhancement device in accordance with aspects of the invention, that is also partially cutaway in selected locations to show details, and in which an admix container is mounted upon a support base and has an air flow control unit for providing pressurized and heated breathable air at the top left as well as the output inhalation mask at the top right;

FIGS. 3 and 4 are partially cutaway side views of alternative configurations for the admix container of FIG. 2 in which the medicinal substance is loose in the container of FIG. 3 and an extension tube for the input air source is shown extending to a position near the bottom of the container, and the admix container in FIG. 4 is shown as containing two separate air-permeable bags in a side-by-side configuration, each bag containing a medicinal substance, which may be the same as in the other bag, or it may be different;

FIG. 5 is a perspective view looking towards the top and side of a third embodiment of a breathing enhancement device in accordance with aspects of the invention in which an air flow controller is at the bottom, an admix container is in the middle, and the user face mask for output infused breathable air is at the top right;

FIG. 6 illustrates a method for applying a layer of a medicinal essential oil onto the outside surfaces of large salt crystals for use in the admix container in accordance with the invention;

FIG. 7 is an exaggerated illustration of applying a layer of essential oil to an internal surface of an admix container, the container being formed of a natural material, in this embodiment a coconut shell, and in which loose or bagged salt crystals will be located;

FIG. 8 is a flowchart illustrating an embodiment of an enhanced breathing method in accordance with aspects of the invention; and

FIG. 9 is a view of a right circular cylinder used as an embodiment of an admix container in accordance with aspects of the invention.

### DETAILED DESCRIPTION OF PREFERRED EMBODIMENTS

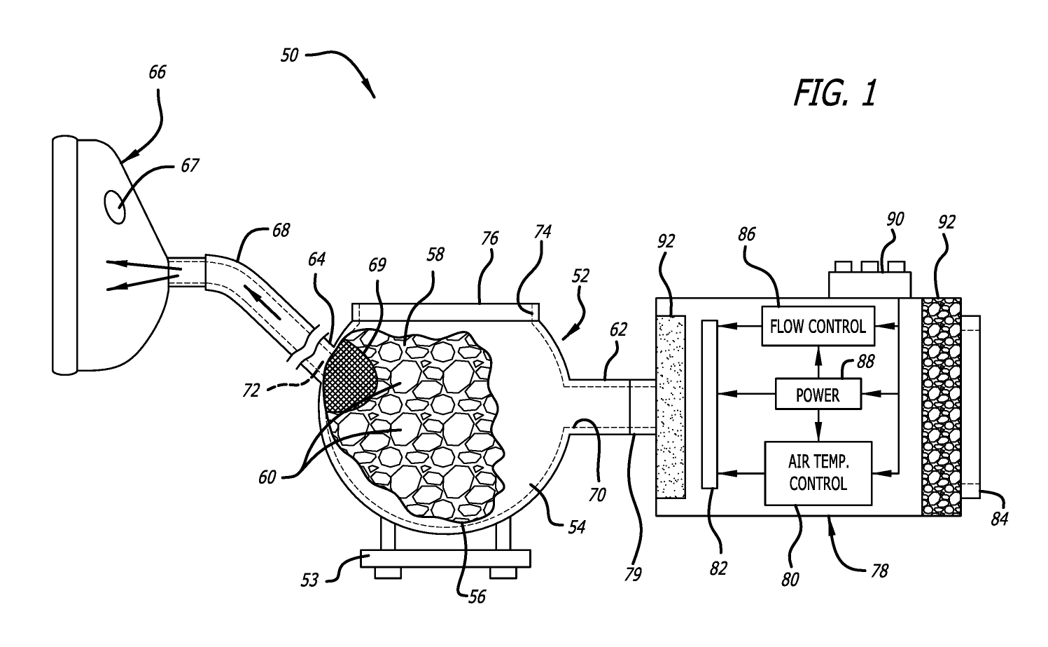

Referring now in more detail to the exemplary drawings for purposes of illustrating embodiments of the invention, wherein like reference numerals designate corresponding or like elements among the several views, there is shown in FIG. 1 a cutaway view of a first embodiment of a breathing enhancement device 50 configured for administering infused air into the lungs of a user. The breathing enhancement device includes a container 52 that rests upon a support base 53. The container has an outer surface 54 and an inner surface 56 that defines an inner cavity 58. Both the inner cavity and the outer surface of the container are generally spherical in shape in this embodiment. The inner cavity is sized (also referred herein as “configured”) for receiving a medicinal substance or substances that may include for example, a plurality of salt crystals 60. In FIG. 1, lead lines from the drawing numeral 60 only point to three salt crystals in the cutaway portion of the container 58 instead of all salt crystals; however, it is intended that drawing reference numeral 60 refer to all salt crystals in FIG. 1. Lead lines from the drawing reference numeral 60 to the other salt crystals were left off to preserve clarity in the illustration).

The container 52 includes an input port 62 for directing breathing air into the inner cavity 58 and an output port 64 for directing infused air out of the inner cavity. If the container has a plurality of salt crystals 60 in the cavity as is shown in FIG. 1, the infused air is referred to as salt-infused air. A vented inhalation mask 66 is connected to the output port 64 by a hollow connection tube 68. The vented inhalation mask is configured to receive the infused air flowing out of the inner cavity of the container 50 via the hollow connection tube. The vented inhalation mask allows a user to inhale the air from the container and to breathe out through the vent 67 in the mask even in the case where there exists pressurized infused air coming into the mask. In this embodiment, a sieve 69 is disposed between the inner cavity and the output port to retain the medicinal substance from falling out of the inner cavity. In another embodiment, a sieve may not be used.

The input port 62 defines an opening 70 that is larger than an opening 72 defined by the output port 64. This difference in sizes with the input port being able to conduct more breathing air into the cavity 58 than the output port can conduct out will increase the resistance to flow of the pressurized breathable air through the inner cavity, which is intended to result in greater circulation of the pressurized input air through the medicinal substance 60 placed within the inner cavity 58 prior to the pressurized air being expelled through the output port and to the vented inhalation mask 66 for inhalation by a user.

The locations of the input port 62 and the output port 64 are removed from each other in this embodiment to promote greater circulation of pressurized breathable input air through the medicinal substance of the inner cavity 58 before the pressurized air is expelled from the inner cavity 58 and through the output port 64 to the vented inhalation mask 66. However, the locations of the input port 62 and the output port 64 may be different than that shown in FIG. 1. It has been found that providing a structure and configuration that causes a greater circulation of the breathing air around and through the medicinal substance or substances residing in the cavity results in greater consistency of the infusion of those medicinal substances into the breathing air. Repeatable concentration of the medicinal substance infusion is more likely to the benefit of the user. The user can be confidant that from one treatment to the next, the concentration will be identical or almost identical.

The breathing enhancement device 50 defines an opening 74 at the top of the container 52 that extends from the inner cavity 58 and through the outer surface 54 of the container 52. The opening 74 is sized such that a medicinal substance may be placed into the inner cavity 58 through the opening 74. In the embodiment of FIG. 1, the opening 74 is sized to be large enough so that a fluid-permeable bag (discussed in further detail below) that contains salt crystals 60 or other medicinal substance can be placed into and removed from the inner cavity through the opening. The enhanced breathing device also includes a cover 76 that is removably positioned over the opening 74 to seal it. The cover is configured such that when it is in place over the opening, it is configured to fit in relation to the opening to seal the opening so that it resists the passage of pressurized breathable air out of the inner cavity 58 through the opening 74.

Continuing to refer to FIG. 1, an air flow control unit 78 of the breathing enhancement device 50 is illustrated. The air flow control unit in this embodiment is configured to draw in breathable air, to pressurize and heat the drawn breathable air, to filter the drawn air, and to move it to the container 52. The air flow control unit has a pressurized air output port 79 that is connected to the input port 62 of the container. The pressurized breathable air exiting the air flow control unit through the pressurized air output port is introduced into the inner cavity 58 of the container 52 and circulated through the contents in the cavity (e.g., the salt crystals 60 or any other medicinal substance disposed within the inner cavity). The pressurized breathable air circulates among the contents disposed within the inner cavity where the air becomes infused with the medicinal substance within the inner cavity and then is expelled out of the inner cavity 58 through the output port 64 and to the vented inhalation mask 66 for inhalation by the user.

The air flow control unit 78 includes an air temperature control device 80 that is configured to adjust the temperature of the breathable air drawn into the air flow control unit 78 through the air input port 84. In one embodiment, the air temperature device comprises a heater that can be set to warm the air to a hyperthermia level. The heater may be provided in different ways, one of which it to use an electrical resistance coil to heat the air as it passes. In another embodiment, a heating/cooling device, such as a thermoelectric cooler, is positioned to heat or cool the air.

The air flow control unit 78 of this embodiment also includes an air mover 82, such as a fan or a blower that draws breathable air into the air flow control unit through the input port 84, pressurizes the drawn air, and moves that pressurized and temperature-controlled air to the pressurized air output port 79. A flow controller 86 is configured to adjust the output of the air mover 82 to either increase or decrease the pressure of the air entering the pressurized air output port 79. A power source 88 (such as a battery) is configured to provide electrical power to the air temperature control device 80, the air mover 82, and the flow controller 86. Electrical switches or control interfaces 90 are configured to power ON or power OFF the air flow control unit 78, to adjust the output and pressure of the air mover 82 via the flow controller 86, and to adjust the air temperature by the air temperature control device 80. One or more filters 92 are disposed at either or both of the input and the output ports 84 and 79 of the air flow control unit. The one or more filters may be any type of filter that facilitates purifying air, such as a charcoal filter. In another embodiment, no filters are used.

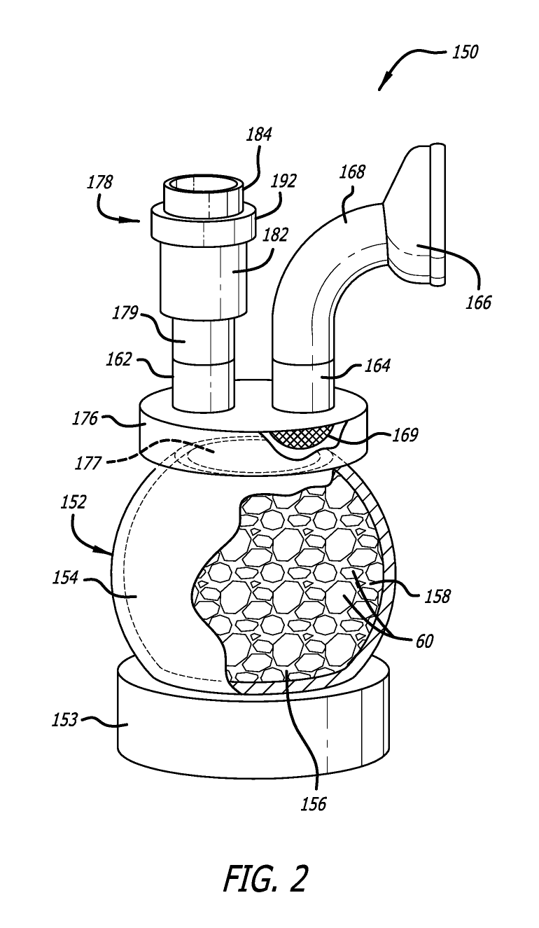

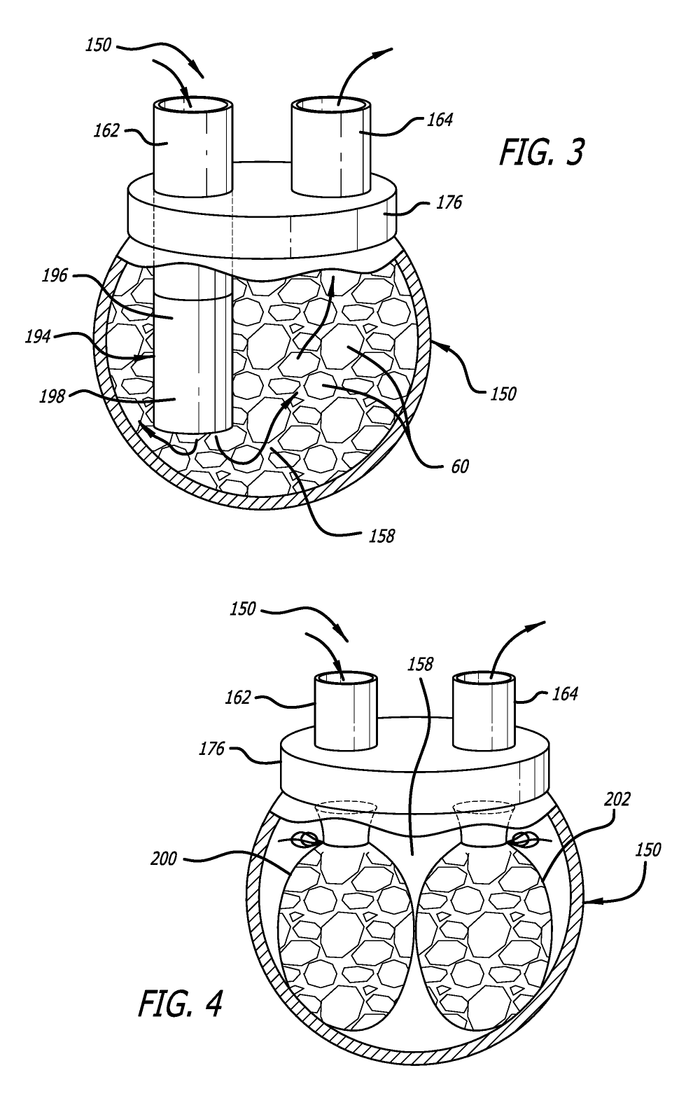

Referring now to FIG. 2, a side partially cutaway view of a second embodiment of a breathing enhancement device 150 in accordance with aspects of the invention is illustrated. The elements of the second embodiment of the breathing enhancement device 150 will have the same functionality as the corresponding elements in the first embodiment of a breathing enhancement device 50 (FIG. 1) unless otherwise stated herein. The second embodiment of the breathing enhancement device 150 includes a container 152, a base 153, an outside surface 154 of the container, an inner surface 156 of the container, an inner cavity 158 defined by the container, an input port 162 into the inner cavity, an output port 164 out of the inner cavity, a vented inhalation mask 166, a hollow output connection tube 168, a sieve 169, a cover 176 positioned over an opening 177, an air pressurization device 178, a pressurized output port 179 from the air pressurization device, an air mover 182, an input port 184 to the air pressurization device, one or more filters 192 associated with the air pressurization device, an air temperature controller (not shown) located in the air pressure control device, and any additional corresponding element for each element of the first embodiment of the breathing device 50 not depicted in FIG. 3. The major difference between the first embodiment of the breathing device 50 (FIG. 1) and the second embodiment of the breathing device 150 (FIG. 2) is that the input port 162 to the inner cavity 158 and the output port 164 from the inner cavity to the vented inhalation mask 166 both extend through the cover 176 in FIG. 2 to established fluid communication with the inner cavity 158 in the second embodiment of the breathing device 150.

Referring now to FIGS. 3 and 4, partial cutaway side views of alternative configurations for the second embodiment of the breathing enhancement device 150 are illustrated. The first configuration of the breathing device 150 depicted in FIG. 3 further includes a hollow air circulation tube 194 having a first end 196, a second end 198, and a length. The first end 196 of the hollow air circulation tube 194 is connected within the inner cavity 158 to the container input port 162 in order to receive the pressurized air. The hollow air circulation tube 194 has a length selected to position the second end 198 of the hollow air circulation tube 194 farther into the cavity than the output port 164. By utilizing the configuration in FIG. 3, the pressurized air is introduced into the inner cavity 158 via the input port 162 at a location deeper within the inner cavity 158 by the hollow air circulation tube 194 than the location of the output port 164 so that the pressurized air is circulated through the medicinal substance within the inner cavity 158 prior to reaching the container output port 164 and being channeled to the vented inhalation mask 166 (not shown).

The second configuration of the breathing device 150 depicted in FIG. 4 further includes a first fluid-permeable bag 200 that contains a medicinal substance or a plurality of medicinal substances, such as salt crystals 60 that are treated with an essential oil or oils, as described below. The first fluid-permeable bag, which includes the salt crystals shown in the figure, is then placed within the inner cavity 158 of the container 150. The pressurized breathable air introduced into the inner cavity 158 of the container 150 via the input port 162 circulates through the fluid-permeable bag 200 and among and throughout the salt crystals that are treated with another medicinal substance, such as the essential oil or oils, before being expelled through the output port 164 to the vented inhalation mask 166 for inhalation by the user (not shown in FIG. 3).

A second fluid-permeable bag 202 located in the cavity 158 adjacent the first bag 200 may contain an additional medicinal substance as desired, such as a different type of salt crystals or more of the same as in the first bag, or other. The pressurized breathable air introduced into the inner cavity 158 of the container 150 via the input port 162 also circulates through the second fluid-permeable bag and among and throughout the medicinal substance contained therein before being expelled through the output port 164 to the vented inhalation mask 166 (not shown in this figure) for inhalation by the user. In one embodiment, the first fluid-permeable bag 200 may specifically contain Himalayan pink salt crystals while the second fluid-permeable bag 202 may contain grey French sea salt. The salt crystals of one, or both, bags may have drops of one or more essential oils on them. The first fluid-permeable bag 200 and the second fluid-permeable bag containing 202 may be made from any porous material that allows for the passage of air through the material, such as a breathable fabric, a porous and flexible plastic material, silk, or other materials.

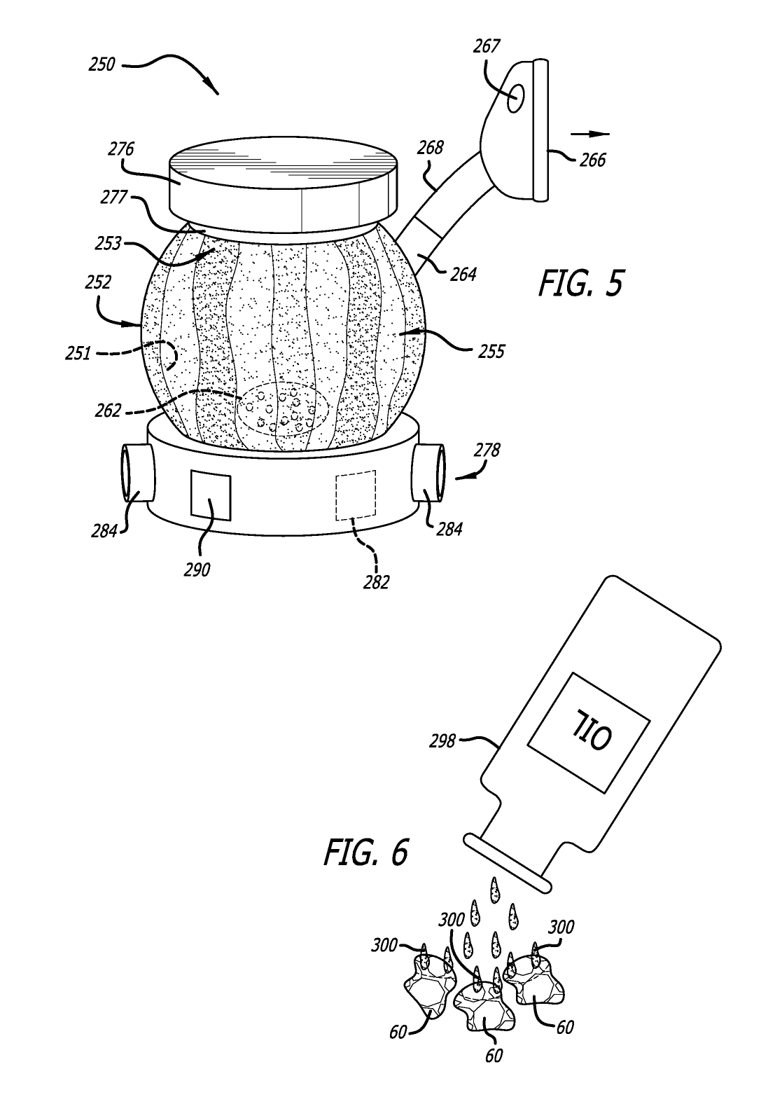

Referring to FIG. 5, side view of a third embodiment of a breathing enhancement device 250 is illustrated. The breathing enhancement device includes corresponding elements and the same functionality with respect to each of the elements of the first and second embodiments of the breathing enhancement device 50 and 150 unless otherwise stated herein. The third embodiment of the breathing device includes a container 252, an input port 262 into an inner cavity 251 of the container 252, an output port 264 out of the inner cavity of the container 252, a vented inhalation mask 266 having at least one vent 267, a hollow output connection tube 268, a cover 276, an air pressurization device 278, an air mover 282, input ports 284 to the air pressurization device 278, electrical switches or control interfaces 290, and any additional corresponding element for each element of the first embodiment of the breathing device 50 not depicted in FIG. 5. The differences between the first embodiment of the breathing device 50 and the third embodiment of the breathing device 250 include the air pressurization device 278 being incorporated into the base structure, the position of the input port 262 to the inner cavity of the container 252 being at the bottom of the container 252, and the container being formed of a coconut shell in the third embodiment of the breathing device 250. It should be noted that containers 50 and 150 in the first and second embodiments may also be formed of coconuts shells.

Coconut shells are plentiful and the shape of the inner cavity of the shell is conducive to thorough air circulation so that the circulated air comes into contact with salt crystals and essential oils that are placed there. The coconut shell is actually the endocarp of the coconut and is a hard, woody layer that is quite strong, yet relatively light. It is attractive in this embodiment because its inner cavity is already formed by nature in a generally spherical shape and no further work is required to form the inside of the container (shell). However, some work must be done to complete the configuration of the outside so that the shell can be put to practical use. As one example, the shell must be given a mount for placing it on a flat surface for stability. As another example, an opening must be made in the shell for insertion and removal of salts and oils and a cover added over the opening to prevent the pressurized breathable air from escaping through the opening.

In one embodiment, the top portion 253 of the coconut is removed to create an opening in the shell through which salt crystals may be inserted or withdrawn from the cavity 255 of the shell. The cover 276 is necessary to seal the coconut so that the pressurized breathing air does not escape from the cavity, except through the output port 264 shown in FIG. 5. In one embodiment, a male thread insert 277 was placed in the top opening of the coconut shell and was permanently held in place with adhesive, such as a non-toxic glue. The cover has an internal seal (not shown) that contacts the threads of the male insert and provides an air tight seal.

Referring now to FIG. 6, a bottle 298 is shown and is being used to apply one or more drops of medicinal oil 300 onto salt crystals 60. The medicinal oil may be any type of medicinal oil or botanical oil including, but not limited to, coconut oil, eucalyptus oil, peppermint oil, lavender oil, clove oil, Ponarus oil, Ayurvedic oil blends, etc. The medicinal oil can be applied to any of the medicinal substances, such as a plurality of salt crystals as shown, which are stored within any of the embodiments of containers 52, 152, or 252 depicted herein. In the present embodiment, eucalyptus oil is shown being applied to the outer surface of salt crystals. Preferably, the oil forms a layer on a salt crystal face or faces, but does not completely cover all the faces so that at least one face will remain open to infuse salt into the pressurized breathing air that is flowing through the cavity of the container and around the salt crystals. Once the pressurized breathable air is introduced into the inner cavity of the container, the pressurized breathable air circulates throughout the cavity and among the medicinal substances placed in the container. The circulating breathable air is infused with the essential oils and the salt before it is expelled through the container output port to the mask for inhalation by the user.

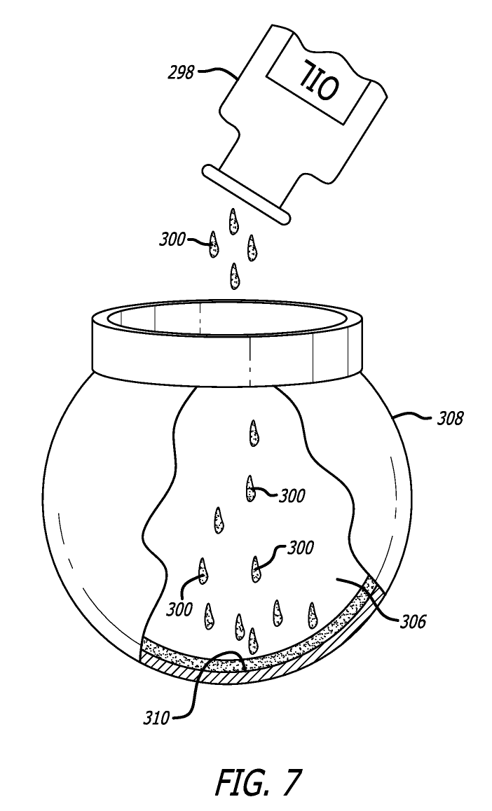

Referring now to FIG. 7, another embodiment is shown in which a medicinal substance 300, such as an essential oil, is located within the cavity 306 of a container 308. In this case, the oil is being applied to an internal surface of a container. The medicinal oil is being applied to coat the internal surface of the cavity within any of the embodiments of containers 52, 152, or 252 depicted herein. However, the shell of the coconut embodiment is especially suited to this process. The inner surface of the coconut shell is rough, as opposed to the smooth surface of a glass container. Essential oils will attach better to the inner surface of a coconut's shell than the internal surface of a smooth glass container. Although not intending to be bound by theory, it has been noted that oil applied to the inner surface of a coconut shell tends to resist gravitational forces that tend to make the oil flow down to the bottom of the coconut shell and pool at the bottom as it does with smooth glass. Having the oil spread around the internal surface of the cavity, as it can be with a coconut, has been found to result in more consistent and repeatable concentrations of that oil in the pressurized breathable air sent to the breathing mask 66.

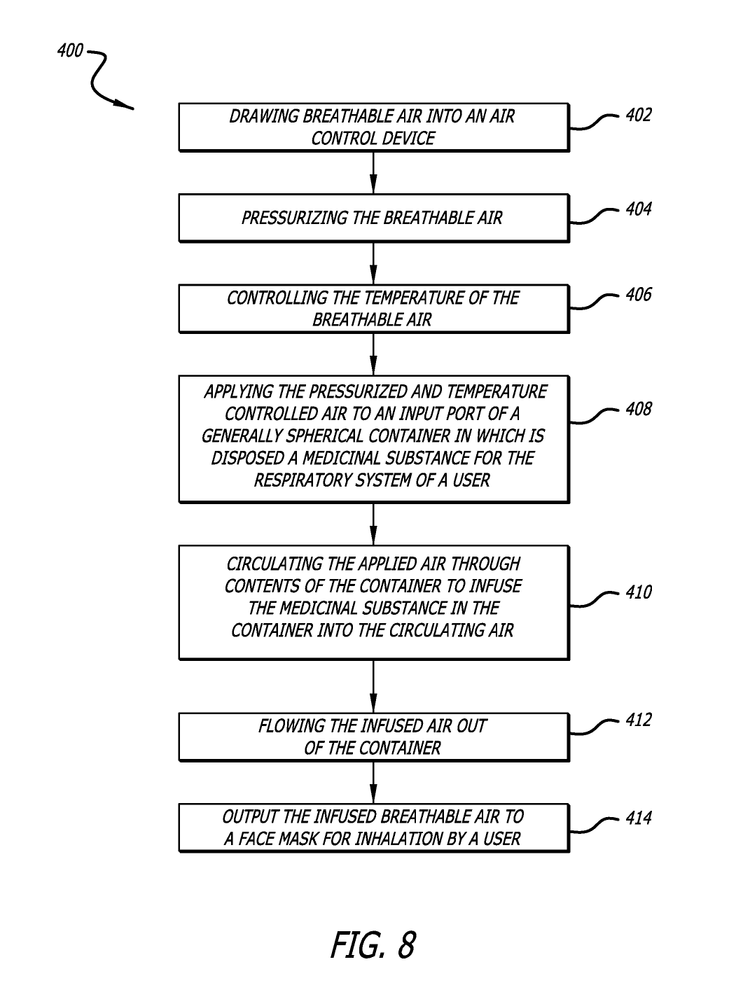

Referring to FIG. 8, a flow chart of a method 400 of providing medicinally-infused breathable air to a user in accordance with aspects of the invention is illustrated. The method 400 begins at block 402 by pressurizing and temperature-controlling breathable air. The pressurized and temperature-controlled breathable air is now circulated 410 through a container in which is located a medicinal substance or substances for being infused into the pressurized and temperature-controlled breathable air as it flows through them. It has been found that spherical or cylindrical containers work well to obtain consistent and repeatable infusions of the medicinal substance or substances into the circulating air. The inventor has also found that such shapes of the internal cavities of containers result in much better flow of the air through the medicinal substance located in the container. The method 400 then moves on to block 412 where the infused air is flowed out of the container and into a face mask for inhalation by a user 414 by the pressurization of the air. As the breathable air arrives at the user's face mask, it has a positive pressure which enables the user to breathe it deeply into his or her lungs with less effort. This air that is forced deeply into the user's lungs has been infused with a medicinal substance or substances, and has been heated or cooled to a desired temperature.

It should be understood that the flowchart in FIG. 8 is for illustrative purposes only and that the method 400 should not be construed as limited to the flowchart in FIG. 8. Some of the steps of the method 400 may be rearranged while others may be omitted entirely.

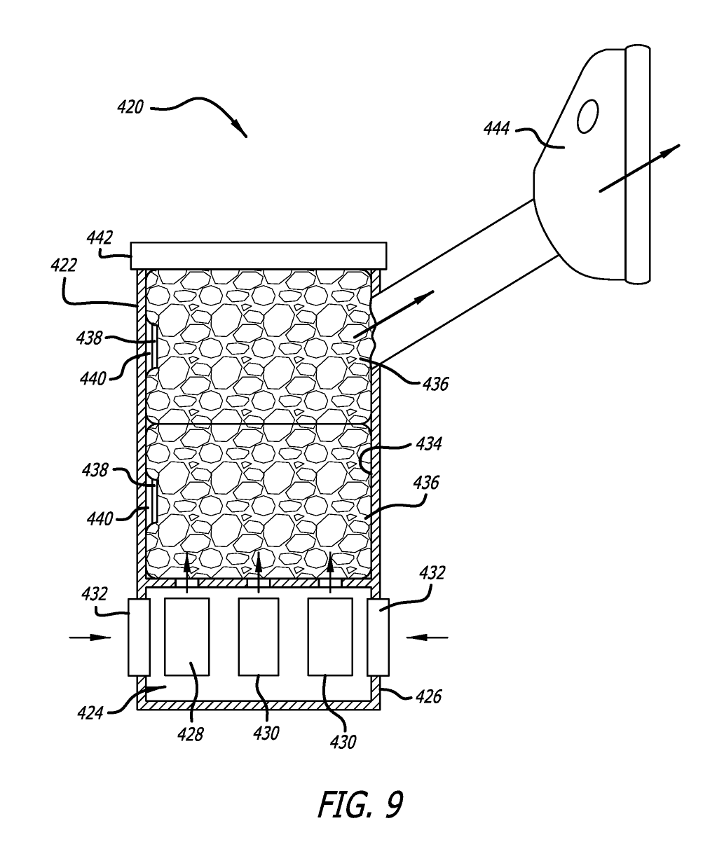

In another embodiment as shown in FIG. 9, another embodiment of an enhanced breathing device in accordance with aspects of the invention is discloses. This embodiment comprises a cylindrical container 422. The air intake 424 is in the base 426 as is the heater 428 and the air pressurizer 430. Ambient air is drawn into the base through filters 432 in this embodiment. The air is heated and pressurized and output upwards into the cavity 434 of the cylindrical container portion. Two bags 436 of salt crystals are shown, each having particular types of salt crystals and at least one of them having botanical oil on the salt crystals. The bags are tied 438 at their open ends 440. A cover 442 allows access to the container for inserting and removing the bags of salt crystals or other medicinal substances that can be used. As in the spherically-shaped cavity, it has been found that an inner cylindrical shape of the cavity in the container such as that shown in FIG. 9 likewise results in a more thorough circulation of the breathable air from an air flow control unit through the medicinal substances in the cavity before the infused air leaves the container of the enhanced breathing device and is delivered to a user's face mask for inhalation.

It is to be understood that the disclosed embodiments are merely examples and other embodiments may take various and alternative forms. The figures are not to scale; some features could be exaggerated or minimized to show details of particular components. Therefore, specific structural and functional details disclosed herein are not to be interpreted as limiting, but merely as a representative embodiment of the invention. As those of ordinary skill in the relevant art will understand, various features illustrated and described with reference to any one of the figures may be combined with features illustrated in one or more other figures to produce embodiments that are not explicitly illustrated or described. The combinations of features illustrated and described herein provide representative embodiments for typical applications. Various combinations and modifications of the features consistent with the teachings herein could be desired for other particular applications or implementations and yet fall within the scope of the invention.

Although described as “spherical,” the inner surface 56 and the inner cavity 58 may not be exactly spherical due to the addition of ports, access holes, and covers. Further, when the container is made of a natural material, such as a coconut shell, the inner cavity is approximately spherical, although it may vary somewhat due to the vagaries of nature. The same is true for the cylindrical container shape.

The embodiments described above include a heater device for controlling the temperature of the breathing air expelled into the face mask. This feature would enable the user to raise the temperature of the breathing air in the case where the user may be suffering from a nasal infection.

As used herein, “medicinal substances” will take many forms without regard to their legality or their acceptance for use by the medical community. Broadly speaking, a “medicinal substance” is anything that tends, or is used, to cure disease or relieve pain. More particularly, it comprises substances that are used for, or have therapeutic properties for curing, healing, and relieving disease.

One medicinal substance is a cannabinoid, even though some cannabinoids are illegal in certain locations. Cannabinoids are derived from the Cannabis genus plant. A species known as Cannabis sativa, or marijuana, is capable of producing psychoactive substances. For example, one of the primary cannabinoids in the marijuana sativa plant is Delta (9)-tetrahydrocannabinol, commonly known as THC. “THC,” or “tetrahydrocannabinol,” is the chemical responsible for most of marijuana's psychoactive effects. Although illegal in many jurisdictions, it has been found to be helpful in treating Alzheimer's disease, neuropathic pain, multiple sclerosis, and Parkinson's disease, along with other diseases, and is therefore a “medicinal substance” as the term is used herein.

The second most famous cannabinoid is cannabidiol or “CBD.” CBD is used as an anti-inflammatory and is therefore a “medicinal substance” as defined herein. The third most famous cannabinoid is Cannabichromene or “CBC” and is used for blocking pain and suppressing nausea and vomiting. It likewise therefore qualifies as a “medicinal substance” herein.

The inventor has found that a spherically-shaped internal cavity provides excellent circulation of the pressurized breathing air through a medicinal substance located in the cavity. The inventor also believes that other symmetrically-shaped cavities may likewise provide improved circulation of the pressurized breathing air for the same purpose.

The words used in the specification are words of description rather than limitation, and it is understood that various changes may be made without departing from the spirit and scope of the disclosure. As previously described, the features of various embodiments may be combined to form further embodiments that may not be explicitly described or illustrated. While various embodiments could have been described as providing advantages or being preferred over other embodiments or prior art implementations with respect to one or more desired characteristics, those of ordinary skill in the art recognize that one or more features or characteristics may be compromised to achieve desired overall system attributes, which depend on the specific application and implementation. As such, embodiments described as less desirable than other embodiments or prior art implementations with respect to one or more characteristics are not outside the scope of the disclosure and may be desirable for particular applications.

Throughout the specification and claims that follow, the word “comprise” and variations thereof, such as “comprises” and “comprising,” are to be construed in an open, inclusive sense, which is as “including, but not limited to.” The meaning of the word “comprising” is to be interpreted as encompassing all the specifically-mentioned features as well optional, additional, unspecified ones. It is to be construed in accordance with the U.S. Patent and Trademark Office Manual of Patent Examination Procedure § 2111.03; i.e., “the transitional term ‘comprising,’ which is synonymous with ‘including,’ containing,' or ‘characterized by,’ is inclusive or open-ended and does not exclude additional, unrecited elements or method steps. ‘Comprising’ is a term of art used in claim language which means that the named elements are essential, but other elements may be added and still form a construct within the scope of the claim.”

While the invention has been described in connection with what is presently considered to be the most practical and preferred embodiments, it is to be understood that the invention is not to be limited to the disclosed embodiments and elements, but, to the contrary, is intended to cover various modifications, combinations of features, equivalent arrangements, and equivalent elements included within the spirit and scope of the appended claims.

What is claimed is:

1. A breathing device for administering medicinally-infused air into the lungs of a user, the breathing device comprising:
a container with an outside surface that defines an inner cavity, and an inner surface within the cavity, the inner cavity being generally spherically-shaped with a size selected for receiving a medicinal substance, the container further comprising separate input and output ports;
a vented inhalation mask having a hollow mask connection tube connected to the container output port to receive air flowing out of the container and conduct the air to the mask for inhalation by a user; and
an air pressurization device configured to receive breathable air and to pressurize the received breathable air, the air pressurization device having a pressurized air output port that is connected to the input port of the container through which the pressurized breathable air is introduced to the inner cavity of the container to circulate the pressurized air through the medicinal substance located within the cavity to infuse the medical substance into the pressurized air as it is circulated through the medicinal substance, and to expel the circulated, breathable, and medicinally-infused air out of the cavity through the output port of the container.

2. The breathing device of claim 1 further comprising a hollow air circulation tube having a first end, a second end, and a length, the first end of the air circulation tube being connected within the cavity to the input port of the container to receive the pressurized air, the air circulation tube having a length selected to position the second end of the circulation tube farther into the cavity than the output port of the container, whereby pressurized air introduced to the cavity at the input port of the container is conducted by the air circulation tube deeper into the cavity than the location of the output port of the container so that the pressurized air is circulated through the medicinal substance located in the cavity before reaching the output port of the container and being expelled to the vented inhalation mask.

3. The breathing device of claim 1 wherein the input port of the container has an opening that is larger than an opening of the output port of the container to cause resistance to flow of the pressurized breathable air through the cavity resulting in greater circulation of the pressurized input air through the medicinal substance in the cavity prior to the pressurized air being expelled through the output port of the container to the inhalation mask for inhalation by a user.

4. The breathing device of claim 1 wherein the locations of the input port of the container and the output port of the container are opposite each other in relation to the cavity.

5. The breathing device of claim 1 further comprising an air temperature control device configured to adjust the temperature of the breathable air that is provided to the input port of the container.

6. The breathing device of claim 5 wherein the air temperature control device comprises a heater positioned to heat the breathable air before the air enters the cavity through the container inlet port.

7. The breathing device of claim 1 further comprising a medicinal substance within the cavity that comprises salt crystals, and wherein the pressurized breathable air introduced through the input port of the container circulates among the salt crystals before being expelled from the cavity through the output port of the container to the inhalation mask for inhalation by the user.

8. The breathing device of claim 7 wherein the medicinal substance within the cavity further comprises a medicinal oil, and wherein the pressurized breathable air introduced to the cavity through the input port of the container is caused to circulate among the salt crystals and the medicinal oil before being expelled through the output port of the container to the inhalation mask for inhalation by the user.

9. The breathing device of claim 1 wherein the medicinal substance within the cavity comprises cannabinoids, and wherein the pressurized breathable air introduced to the cavity through the input port of the container is caused to circulate among the cannabinoids before being expelled through the output port of the container to the inhalation mask for inhalation by the user.

10. The breathing device of claim 7 further comprising a first fluid-permeable bag containing salt crystals, the first bag of salt crystals being located within the cavity of the container wherein the pressurized breathable air introduced through the input port of the container is caused to circulate through the first fluid-permeable bag and among the salt crystals therein before being expelled through the output port of the container to the inhalation mask for inhalation by the user.

11. The breathing device of claim 10 further comprising a second fluid-permeable bag containing a medicinal substance, the second bag of the medicinal substance being located within the cavity of the container adjacent the first bag wherein the pressurized breathable air introduced through the input port of the container circulates through the first fluid-permeable bag comprising salt crystals and through the second fluid-permeable bag of medicinal substance before being expelled through the output port of the container to the mask for inhalation by the user.

12. The breathing device of claim 1 further comprising:
an opening formed through the outer surface of the container, the opening having a size large enough to place the fluid-permeable bag in the cavity and to remove the fluid-permeable bag from the cavity; and
a cover removably positioned over the opening, the cover configured to resist passage of pressurized breathable air out of the cavity through the opening.

13. The breathing device of claim 12 wherein the input and output ports of the container are provided in the cover, and wherein the cover is configured to resist passage of pressurized breathable air out of the cavity through the opening.

14. The breathing device of claim 1 wherein the container is formed of a coconut shell.

15. The breathing device of claim 14 further comprising coconut oil coated onto the inner surface of the cavity of the coconut.

16. A breathing device comprising:
a container having a generally spherically-shaped internal cavity configured to receive an infusible medicinal substance;
the container having an inlet in fluid communication with the spherically-shaped internal cavity and an outlet in fluid communication with the spherically-shaped internal cavity;
a breathing mask externally secured to the container and in fluid communication with the outlet of the cavity; and
an air pressurization device secured to the container and configured to produce pressurized breathable air and to force the pressurized breathable air through the inlet into the spherically-shaped internal cavity to flow across an infusible medicinal substance located in the cavity so that the medicinal substance is infused into the pressurized air flowing across it, and to expel the infused breathable air through the outlet and into the breathing mask.

17. A method of providing medicinally-infused breathable air, comprising:
pressurizing breathable air;
applying the pressurized breathable air to an input port of a container that has a spherically-shaped interior cavity in which is located an infusible medicinal substance;
flowing the pressurized breathable air through the input port and across the medicinal substance to infuse the flowing pressurized breathable air with the medicinal substance; and
expelling the infused pressurized breathable air from the cavity through an output port of the cavity;
directing the expelled infused pressurized breathable air to a vented face mask.

18. The method of claim 17 further comprising heating the breathable air prior to applying the pressurized breathable air to the input port of the container.

19. The method of claim 17 further comprising flowing the pressurized breathable air across salt crystals located in the spherically-shaped interior cavity of the container.

20. The method of claim 17 further comprising disposing a medicinal substance within a fluid-permeable bag and then locating the fluid permeable bag within the spherically-shaped interior cavity.
